(19) United States
(12) Patent Application Publication
(10) Pub. No.: US 2006/0159744 A1
(43) Pub. Date: Jul. 20, 2006

(54) Timed dual release dosage forms comprising a short acting hypnotic or a salt thereof

(75) Inventors: Gerard Alaux, Beynes (FR); Frederic Andre, Antony (FR); Jean Ducassou, Anglet (FR); Gareth Lewis, Dourdan (FR)

Correspondence Address: ROSS J. OEHLER;AVENTIS PHARMACEUTICALS INC., 1041 ROUTE 202-206, MAIL CODE: D303A, BRIDGEWATER, NJ, 08807, US

(73) Assignee: Sanofi-Aventis, Paris (FR)

(21) Appl. No.: 11/385,408

(22) Filed: Mar. 21, 2006

Related U.S. Application Data
(63) Continuation of application No. 10/818,666, filed on Apr. 6, 2004.
Application No. 10/818,666 is a continuation of application No. 10/019,726, filed on Dec. 20, 2001, now abandoned.

(30) Foreign Application Priority Data
Jun. 28, 1999 (EP) 99401605.3

(51) Int. Cl. A61K 9/48; A61K 9/26

(52) U.S. Cl. 424/451; 424/469

(57) ABSTRACT

The present invention relates to timed dual release dosage forms of short acting hypnotics or salts thereof adapted to release the short acting hypnotic over a predetermined time period, according to a profile of dissolution characterized in that it comprises two release pulses, the first being immediate (lasting up to 30 minutes) and the second being delayed by a fixed time (this fixed time being between 50 and 200 minutes).

### CROSS REFERENCE TO RELATED APPLICATIONS

This application is a continuation of U.S. application Ser. No. 10/818,666, filed Apr. 6, 2004, now pending, which is a continuation of Ser. No. 10/019,726, filed Dec. 20, 2001, abandoned, which was the National Stage of International application No. PCT/EP00/06,792, filed Jun. 27, 2000; all of which are incorporated herein by reference in their entirety, which claims the benefit of priority of European Patent Application No. 99 401605.3, filed Jun. 28, 1999.

The present invention relates to timed dual release dosage forms comprising short acting hypnotic or salts thereof.

The short acting hypnotic can belong to all therapeutic classes:
compounds of the therapeutic class of pyrazolopyrimidines, such as zaleplon,
compounds of the therapeutic class of cyclopyrrolones, such as zopiclone and its enantiomers like (R)-zopiclone,
compounds of the therapeutic class of benzodiazepines, such as triazolam, temazepam or brotizolam,
compounds of the therapeutic class of phenothiazines, such as is alimemazine or the tartrate thereof,
compounds of the therapeutic class of imidazopyridines, such as zolpidem. One of the preferred salts of zolpidem is zolpidem hemitartrate.

Up to now, according to the rapidity of action of this kind of active substances, only immediate release dosage forms were developed, which disintegrate rapidly in the gastrointestinal tract, dissolve in the fluid of the gastrointestinal tract and undergo systemic absorption, where the short acting hypnotic, which we will call hereinafter “the drug”, can exert its pharmacological effect and induce sleep of the patient.

The new dosage forms according to the present invention enable first of all a sufficient blood level of short acting hypnotic to be obtained rapidly after administration in order to induce sleep, and then a second pulse of short acting hypnotic to be released after a fixed time after administration in order to maintain sleep.

Therefore, as a first object, the present invention provides timed dual release dosage forms comprising short acting hypnotics or salts thereof adapted to release over a predetermined time period, according to a profile of dissolution, characterized in that it comprises two release pulses, the first being immediate and the second being delayed to a fixed time.

The “total amount of drug” means the quantity by weight of the drug comprised in the whole dosage form according to the invention.

The immediate release portion of the profile (initial pulse) is defined as that proportion of the drug dissolved in 30 minutes in a suitable in vitro dissolution test. A suitable dissolution test is for example one of the methods described in example 1: method where measurement is carried out with the rotating paddle apparatus of the European pharmacopoeia, at a stirring speed of 50 rpm, in an aqueous buffer at pH between 1 and 7.5 at 37° C., or variations on this as well known to one skilled in the art.

The proportion of the drug dissolved during this pulse is the proportion of the total amount of the drug which is dissolved at 30 minutes. In an advantageous embodiment of the dosage forms according to the present invention 90% or more of that part of the drug allotted for the initial pulse is dissolved in 20 minutes and more preferably in 15 minutes. This embodiment is particularly advantageous for dosage forms comprising zolpidem or a salt thereof.

The delayed release portion of the profile is the part of the dissolution occurring after 30 minutes, measured in a suitable in vitro dissolution test, such as described in example 1.

The delayed release portion of the profile is defined by the percentage released at times T₁ and T₂, defined as follows.

T₁ describes the beginning of the second, delayed release pulse, and is defined as the time for release of 10% of the drug allotted for the delayed release portion of the profile.

T₂ describes the end of the delayed release pulse, and is defined as the time for release of 85% of the drug allotted for the delayed release portion of the profile.

The release of the delayed release pulse may be less rapid than the immediate release pulse. For example, the period (T₂-T₁) can last between 30 and 200 minutes.

Moreover, the delayed release pulse can begin between 50 minutes and 200 minutes after the beginning of dissolution, and preferably between 60 and 150 minutes, this range of time being defined as the “fixed time”.

Indeed, the delayed release should be completed at a time after administration compatible with the desired time of sleep, and the time needed for elimination of the drug from the human body to a sufficiently low level roughly 8 hours after administration. In view of this, T₂ is between 2 and 6 hours and preferably between 2.5 and 5 hours.

The immediate release pulse can liberate between 40 to 70% of the total amount of the drug.

An example of such an in vitro release profile is given in FIG. 1, where 60% of the total amount of drug is released during the immediate release pulse, and the second, delayed release pulse occurs after 90 minutes (T₁) with a T₂ time being equal to 150 minutes.

As a second object, the present invention provides timed dual release dosage forms of short acting hypnotics or salts thereof, characterized in that they comprise two kinds of pharmaceutical entities of drug: one immediate release entity and one delayed release entity. The drug dissolved during the initial, immediate release pulse (before 30 minutes) is contained within the immediate release entity, and that liberated in the second, delayed release pulse (beginning after the fixed time) is contained within the delayed release entity.

Small quantities of the drug in a formulation for rapid release can be retained in the formulation and thus may be released at a time after 30 minutes from the beginning of the dissolution, and are thus included in the delayed release part of the profile. Similarly, small quantities of the drug incorporated in the delayed release pharmaceutical entity may be released before 30 minutes, and thus form part of the immediate release part of the profile.

According to the present invention, the proportion of the drug contained within the immediate release entity and dissolved within 30 minutes is at least 90%. And the proportion of the drug contained within the delayed release entity and released within 30 minutes is comprised between 0 and 20%, and preferably between 0 and 5%.

Among dosage forms able to match the requirement of a timed dual release profile and to comprise the two kinds of pharmaceutical entities defined above, the following may be cited: capsules, tablets, multilayer tablets, multicoated tablets.

The immediate release entity shall be understood in the present invention as a single pharmaceutical immediate release unit like for example an immediate release tablet or pellet, or several such units formulated into a capsule or a tablet; as an immediate release matrix in a tablet; as an immediate release layer, that can be incorporated in a multilayer tablet; as an immediate release coating layer in a multicoated tablet or pellet.

The delayed release entity shall be understood in the present invention as a pharmaceutical delayed release unit such as, for example, a delayed release tablet or pellet, or several such units formulated into a capsule or a tablet; as a delayed release core or a delayed release coating layer in a multicoated tablet; as delayed release pellets within a disintegrating tablet.

Dosage forms where the immediate release entity and the delayed release entity are administered simultaneously but separately are also encompassed in the present invention.

The total amount of short acting hypnotic contained in the dosage forms according to the present invention depends on the individual drug.

For example, the dosage forms according to the invention typically contain from 10 to 30 mg of zaleplon or from 7.0 to 15 mg of zopiclone.

In the same way, the dosage forms according to the invention typically contain from 4 to 16 mg of zolpidem as zolpidem base, and preferably 6 to 12 mg of zolpidem as zolpidem base. The zolpidem may be incorporated as the base, or as a pharmaceutically acceptable salt of zolpidem. Among dosage forms comprising a salt of zolpidem rather than the zolpidem base, according to the invention, those comprising zolpidem hemitartrate are especially preferred.

In advantageous embodiments, dosage forms may be formulated in order to obtain a dissolution independent of the pH in the second release pulse. The preferred manner to achieve such a dissolution in the case of a basic short acting hypnotic, like zolpidem, zopiclone or zaleplon, is to add a pharmaceutically acceptable organic acid into the dosage form, according to methods known from one skilled in the art. Such dosage forms are preferred.

These pharmaceutically acceptable organic acids can be chosen for example among maleic, tartaric, malic, fumaric, lactic, citric, adipic or succinic acid and their acid salts where these exist, in the form of racemates or isomers, where these exist. According to the invention, acids particularly preferred are tartaric, fumaric, citric, and succinic and their acid salts.

Various formulations, not limiting the scope of the present invention, illustrating the invention are described hereafter:

(1) A mixture of immediate release and delayed release particles of dimension 0.2-2 mm, known variously as pellets, beads, granules or spheroids, within a capsule:

The beads, pellets, granules or spheroids may be manufactured by any of the methods well known to one skilled in the art: granulation in a high speed granulator, extrusion followed by spheronization, gradual coating of a sugar sphere consisting of sugar or microcrystalline cellulose or mannitol or any other suitable pharmacologically inert substance, with a mixture containing the drug etc.

A part of the pellets, granules or spheroids is then coated for delayed release as described hereinafter. The coating must be impermeable to the drug on contact with aqueous fluid, but becomes permeable to the drug after a suitable period as described above, and not earlier, as a result either of erosion of the coating, or by increase in permeability of the coating, for example by the formation of aqueous pores, or by breakdown of the film, which may be obtained by means of:

(i) a coating which contains one or more polymers impermeable to water and to drug molecules, such as ethylcellulose, ammonio methacrylate copolymer Type B, cellulose acetate, cellulose acetate butyrate, polyvinyl chloride, polyvinylacetate and one or more polymers which are permeable to water, such as hydroxypropylmethyl-cellulose, hydroxyethylcellulose, methylcellulose ammonio methacrylate copolymer Type A, the composition of the mixture being adjusted to allow gradual hydration of the film and a delayed release dissolution profile.

(ii) a coating containing a mixture of polymers as in (i) which are physically incompatible with one another (immiscible). An example of such a mixture is that of ethylcellulose and methacrylate copolymers with quaternary ammonium groups (ammonio methacrylate copolymer Type A or B).

(iii) a hydrophobic erodible coating, consisting of a wax such as carnauba wax, glyceryl behenate, or hydrogenated castor oil. This may be mixed with one or more insoluble diluents such as calcium dihydrogen phosphate or talc. It can be applied as the melted wax, for example in a fluid-bed coating apparatus.

According to a preferred embodiment, in the case where one or more of the coating polymers is an ammonio methacrylate copolymer, a suitable cationic surfactant, or an amphoteric or zwitterionic surfactant is added into the core.

The surfactant diffuses into the coating, and at a given level provokes a sudden change in the films properties, provoking a sudden rapid release.

This particular embodiment presents the advantage that the delayed pulse is accelerated and gives substantially more complete release of the active substance than for pellets, granules or spheroids coated with methacrylate copolymer without surfactants in their cores.

Examples of such cationic surfactants are trimethyl-dimyristoyl-ammonium propane, dimethyl-dioctadecyl-ammonium bromide, trimethyl-cetyl-ammonium bromide (CTAB), dimethyl-didodecyl-ammonium bromide (DDAB(12)), benzalkonium chloride, cetylpyridinium chloride, and cetramide.

Examples of zwitterionic surfactants are the N-alkylbetaines, the C-alkylbetaines, the N-alkylamidobetaines such as cocamidopropylbetaine, the N-alkylglycines and the phosphatidylcholines or lecithins.

This method is employed in the case of examples 2, 3 and 5.

The said preferred formulation where one or more of the coating polymers is an ammonio methacrylate copolymer can also contain a mixture of cationic and/or zwitterionic surfactants especially mixtures of the aforementioned surfactants.

In the case of the coating being a hydrophobic wax, one or more non-ionic surfactants may be included in the formulation, in the core, in order to promote dissolution and erosion of the film.

The core may contain other substances known to be necessary or advantageous to formation by one skilled in the art of pharmaceutical formulation, in particular an organic acid to maintain the pH at the interior of the pellet constant. The core may also be coated with a water-soluble polymer, for example hydroxypropylmethylcellulose or polyvinylpyrrolidone, before application of the outer coating, in order to eliminate contact between the core and the outer coating.

(2) A mixture of delayed release particles and an immediate release powder, within a capsule:

The delayed release particles, known variously as granulates, pellets, beads, microspheres are those described above in (1). The immediate release powder Is prepared by a simple mixture of the drug with pharmaceutically inactive substances, or by granulation of a mixture of drug mixed with pharmaceutically inactive substances, using one of the granulation methods well known to one skilled in the art of pharmaceutical formulation.

(3) A tablet containing delayed release coated pellets as described in (1) containing the drug imbedded in a matrix also containing the drug.

Alternatively the tablet may consist of a mixture of delayed release coated pellets and of immediate release non-coated pellets containing the drug, imbedded in a matrix free from the drug.

Alternatively the delayed release coated pellets may be furthermore coated with a layer containing the drug and other excipients allowing immediate release from that layer, imbedded in a matrix free from the drug.

Alternatively the tablet may consist of one or more layers containing delayed release coated pellets containing the drug, imbedded in a matrix free from the drug and one or more layers containing the drug in an immediate release matrix.

The matrix surrounding the pellets should preferably be formulated so that the compression into tablets does not interfere with the integrity of the membrane surrounding the pellets. On contact with fluid the tablet disintegrates, releasing the drug rapidly, from the matrix, or the immediate release pellets, or from the immediate release pellet coating, or from the immediate release layer, and then, after a set interval of time, releasing the drug from the delayed release pellets. The pellet may be formulated with a pharmaceutically acceptable organic acid so as to maintain the micro-pH of the pellet during dissolution in the neutral pH conditions. The matrix consists of inert pharmaceutical substances such as well known to one skilled in the art of pharmaceutical formulation. In particular the matrix includes one or more diluents such as microcrystalline cellulose, lactose, mannitol, starch and one or more disintegrants, for example crospovidone, sodium starch glycolate and croscarmellose. Other excipients may also be included, lubricants, for example magnesium stearate, glyceryl stearate, and glyceryl behenate, binders, for example hydroxypropylmethyl-cellulose, ethylcellulose and povidone, glidants, for example talc and colloidal silicon dioxide.

(4) A capsule containing one or more immediate release tablets and one or more delayed release tablets.

The immediate release tablet or tablets may be formulated by the methods well known to one skilled in the art. In addition to the drug they can contain inert pharmaceutical excipients, including one or more diluents, for example microcrystalline cellulose, lactose, mannitol, starch; and may contain other excipients. These can include one or more binders, for example hydroxypropylmethylcellulose, ethylcellulose and povidone, lubricants, for example magnesium stearate, glyceryl stearate, and glyceryl behenate, disintegrants, for example crospovidone, sodium starch glycolate and croscarmellose, glidants, for example talc and colloidal silicon dioxide. The cores of the delayed release tablets may be prepared using the same excipients as the immediate release tablets except that additional substances may be added. In particular a pharmaceutically acceptable acid may be added to ensure liberation of the drug independent of the pH of the external medium.

The delayed release tablets are coated with a layer of polymer coating similar to those described for the multiparticulate pellet systems above. However some modification of the coating will be required because of the difference in surface area of the dosage form. It is usually necessary to apply a thicker coating on the tablet than on the pellets, and thus a higher proportion of water-permeable polymers will be required in the coating composition.

In the case of a hydrophobic wax coating, the wax may be mixed with a soluble diluent such as polyethylene glycol, and the mixture applied by press coating.

In the case of coatings containing a methacrylate copolymer, a cationic surfactant may advantageously be included within the delayed release tablet core.

In the case of coatings containing a hydrophobic, waxy excipient such as carnauba wax or hydrogenated castor oil, a non-ionic surfactant may be included within the tablet core. The tablet core and the coating may also be separated by a coating of a water-soluble polymer, for example hydroxypropylmethylcellulose or polyvinylpyrrolidone.

As other particular embodiments encompassed within the scope of the present invention, pharmaceutical compositions intended to avoid abuse may be cited.

Indeed it is known that some drugs and in particular hypnotics intended for legitimate oral use have the potential for abuse.

One way of substantially reducing or even eliminating this potential for drug abuse for the pharmaceutical formulations that are objects of the present invention is to provide pharmaceutical compositions for oral administration comprising a short acting hypnotic or a salt thereof capable at the same time of:

liberating the active principle according to a timed dual release in vitro profile as described above, following normal administration and,

if it is introduced in a drink, whether or not containing alcohol, generating visual change or changes in the appearance of the drink. This visual change or changes are intended to avoid administration of the active principle to a person in the said drink without his or her knowledge.

These visual changes, according to the present invention, include all means of indicating the presence of the said composition in a drink. The following may be cited as methods for inducing visual changes: inclusion of coloring excipients, floating of the composition at the surface of the drink, formation of insoluble particles on the surface of the drink, on the brim of the glass, in the drink and/or on the bottom of the glass or a combination thereof.

The drink, eventually with alcohol, may for example consist of coffee, tea, wine, fortified wine, spirits, liqueurs, hot or cold chocolate-flavored drinks, all gaseous alcoholic or not-alcoholic drinks, all cocktails or mixtures of fruit juice, milk, cream, . . .

Particles may be obtained by association of a lipophilic and a hydrophilic excipient, useful for the floating as described above. A list of suitable lipophilic excipients is set forth beneath.

The composition according to this particular embodiment of the present invention can liberate particles even if the composition does not float or not immediately.

Among lipophilic excipients the following may be cited: glycerol stearates, palmitostearates and behenates; hydrogenated vegetable oils and their derivatives; vegetable and animal wax and their derivatives; hydrogenated castor oils and their derivatives and cetylic esters and alcohols.

Among hydrophilic excipients the following may be cited: cellulose derivatives, hydroxyethylcellulose, hydroxypropylcellulose (molecular mass from 50 to 1250 kDa), hydroxypropylmethylcellulose (molecular mass from 10 to 1500 kDa), carboxymethylcellulose and sodium carboxymethylcellulose; vegetable gums and their derivatives; derivatives of alginic acid; polyethyleneglycols and their derivatives; starches and their derivatives; silica, polymethacrylates and acrylic acid and methacrylate copolymers.

One of the constituents of the gel forming substance can be chosen as being less soluble in alcohol.

A coloring excipient can be advantageously added as giving rise to visual change preventing abuse. It can color simultaneously the liquid or the particles or one independently of the other.

Among suitable coloring excipients the following may be cited: indigotine, cochineal carminic acid, yellow orange S, allure red AC, iron oxides, cucurmin, riboflavin, tartrazine, quinoline yellow, azorubine, amaranth, carmines, erythosine, red 2G, patented blue V, glittering blue FCF, chlorophylls, copper complexes of chlorophylls, green S, caramel, glittering black BN, carbo medicinalis vegetabilis, brown FK and HT, carotenoids, Annatto extracts, paprika extracts, lycopene, lutein, canthaxanthin, beetroot red, anthocyanes, calcium carbonate, titanium dioxide, aluminum, silver, gold or litholrubin BK or any other coloring excipient suitable for an oral administration.

These visual means of preventing abuse may comprise a distinct pharmaceutical entity, not containing active substance, along with the immediate release and the sustained release entities, that comprise the pharmaceutical form, or they may be incorporated in one of these two entities. Yet a third method is to incorporate all or certain of them into a separate entity and at the same time add certain to the immediate or sustained release entity.

The method of incorporation of abuse resistance as described above will depend on the type of formulation. In the case of tablet formulations described above, including that of tablets enclosed inside a capsule, the abuse resistance conferring substances (coloring matter, effervescent couple . . . ) may be included within the immediate release entity of the formulation.

Alternatively in the case of multilayer tablets and immediate tablets within a capsule they may be incorporated as a separate layer not containing active substance, but with the abuse resistance conferring substances. Such a layer may be added to the sustained release tablet or tablets within a capsule provided the said tablet is formulated as a matrix and is not coated with a coating conferring the sustained release properties.

In the case of a capsule containing controlled release pellets and immediate release pellets or granulate, abuse resistance conferring substances may be incorporated in the immediate release entity or added separately.

### LIST OF FIGURES

FIG. 1 shows an example of a in vitro timed dual release profile, where the immediate release pulse is 60% of the total amount of zolpidem, and the second pulse is 40%, starting at 90 minutes and finishing at 150 minutes.

FIG. 2 shows an in vitro dissolution profile of the uncoated pellets containing zolpidem hemitartrate of example 1 at pH 2.

FIG. 3 shows an in vitro dissolution profile of the coated pellets containing zolpidem hemitartrate of example 2 at pH 2 and 6.8.

FIG. 4 shows an in vitro dissolution profile of the coated pellets containing zolpidem hemitartrate of example 3 at pH 2 and pH 6.8.

FIG. 5 shows the in vitro dissolution profile of a capsule of example 4 containing a mixture of the uncoated pellets of example 1 and the coated delayed release pellets of example 3, containing 7.5 mg zolpidem hemitartrate in each type of pellet, at pH 2.

FIG. 6 shows in vitro release profiles of coated pellets containing zolpidem hemitartrate of comparative example 1 at pH 2 and 6.8.

FIG. 7 shows in vitro release profiles of coated pellets containing zolpidem hemitartrate of comparative example 2 at pH 2 and 6.8.

FIG. 8 shows the in vitro dissolution profile of the coated pellets containing zolpidem tartrate of example 5.

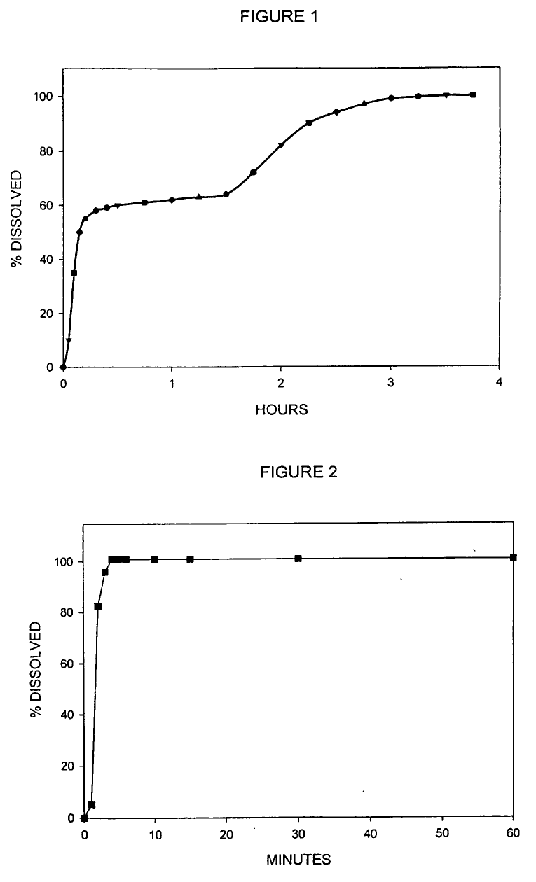

The examples which follow illustrate the invention without limiting it:

### EXAMPLE 1

### Immediate Release Pellets Containing Zolpidem Hemitartrate

1000 g of nonpareil beads 16/18 mesh were coated using a suspension with the following composition,

| | | |
|---|---|---|
| zolpidem hemitartrate | 11.54% | 78.125 g |
| povidone K30 ¹ | 11.54% | 78.125 g |
| Ethanol | 76.92% | 520.8 g |

¹ Kollidon ® commercialized by BASF

The coating was carried out using a GPCG1 fluid bed coated-dryer (Glatt). The dissolution of the beads was measured using the method described in the European pharmacopoeia, with the rotating paddle apparatus, at a stirring speed of 50 rpm. Dissolution medium was 900 ml, 0.01 M hydrochloric acid, at 37±0.5° C. The amount of zolpidem hemitartrate dissolved was measured by UV spectrophotometry at 310 nm. The dissolution curve obtained is shown in FIG. 2.

### EXAMPLE 2

### Coated Pellets

Delayed release pellets containing zolpidem hemitartrate, tartaric acid and benzalkonium chloride as cationic surfactant

1000 g of nonpareil beads 16/18 mesh were coated using a suspension with the following composition,

| | | |
|---|---|---|
| tartaric acid | 6.0% | 78 g |
| Hydroxypropylmethylcellulose¹ | 4.0% | 53 g |
| benzalkonium chloride | 3.0% | 39 g |
| purified water | 43.5% | 567 g |
| isopropanol | 43.5% | 567 g |

¹Pharmacoat ® 603 commercialized by Shin-Etsu

The pellets were then loaded with zolpidem hemitartrate by coating with the following solution, in a GPCG1 fluid bed coater-dryer:

| | | |
|---|---|---|
| zolpidem hemitartrate | 8.3% | 78 g |
| povidone K30 ² | 8.3% | 78 g |
| ethanol | 83.4% | 784 g |

² Kollidon ® commercialized by BASF

Finally the pellets were coated using a polymer solution of the following composition:

| | | |
|---|---|---|
| ammonio methacrylate | 11.40% | 83.4 g |
| copolymer Type B ³ | | |
| ammonio methacrylate | 0.93% | 6.8 g |
| copolymer Type A 4 | | |
| triethyl citrate ⁵ | 1.37% | 10.0 g |
| isopropanol | 51.80% | 379.0 g |
| acetone | 34.50% | 252.0 g |

³ Eudragit ® RS100 commercialized by Rohm Pharma
⁴ Eudragit ® RL100 commercialized by Rohm Pharma
⁵ Eudraflex ® commercialized by Rohm Pharma

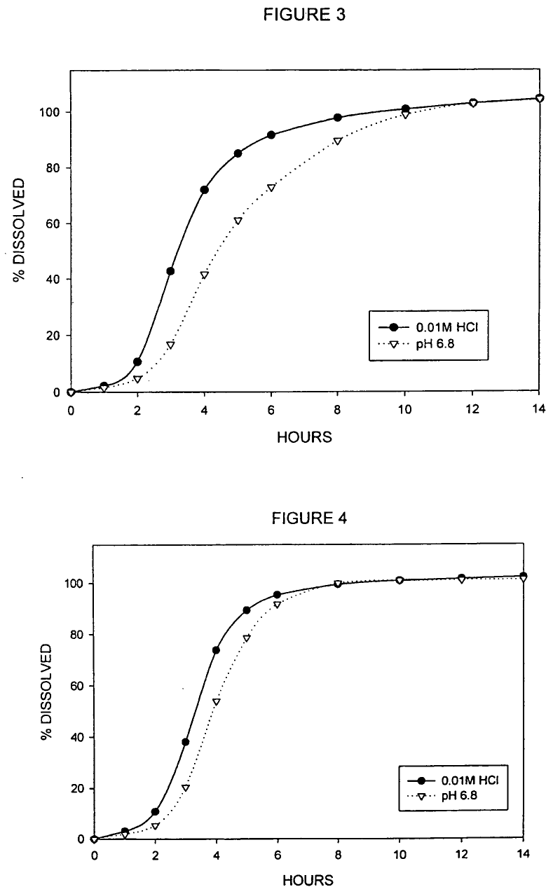

The dissolution profiles of the pellets were tested in 0.01 M hydrochloric acid using the method described in example 1, and in a 0.02M pH 6.8 potassium phosphate buffer solution containing 0.1 M sodium chloride, all other parameters being the same as for the test in hydrochloric acid. They are shown in FIG. 3.

### EXAMPLE 3

### Coated Pellets

Delayed release pellets containing zolpidem hemitartrate, tartaric acid and cetylpyridinium chloride as cationic surfactant

1000 g of nonpareil beads 16/18 mesh were coated using a suspension with the following composition:

| | | |
|---|---|---|
| tartaric acid | 6.0% | 78.0 g |
| Hydroxypropylmethylcellulose ¹ | 4.0% | 53.0 g |
| cetylpyridinium chloride | 3.0% | 39.0 g |
| triethyl citrate ² | 1.4% | 18.2 g |
| purified water | 42.8% | 557.0 g |
| isopropanol | 42.8% | 557.0 g |

1 Pharmacoat ® 603 commercialized by Shin-Etsu
² Eudraflex ® commercialized by Rohm Pharma

The beads were then loaded with zolpidem hemitartrate by coating, in a GPCG1 fluid bed coated-dryer, and finally coated using a polymer solution, using the same methods and compositions as described in example 2. The dissolution profiles of the pellets were measured as described in example 2. These are shown in FIG. 4.

### EXAMPLE 4

### Capsule Containing a Mixture of Immediate Release and Delayed Release Pellets Containing Zolpidem Hemitartrate

Capsules containing 15 mg zolpidem hemitartrate were manufactured according to the following composition

| | | Zolpidem |
|---|---|---|
| Component | Mass per unit | hemitartrate content |
| uncoated beads of | 112 mg | 7.5 mg |
| example 1 | | |
| coated beads of | 131 mg | 7.5 mg |
| example 3 | | |
| hard gelatine | | — |
| capsule size 3 | | |
| (Total) | | 15.0 mg |

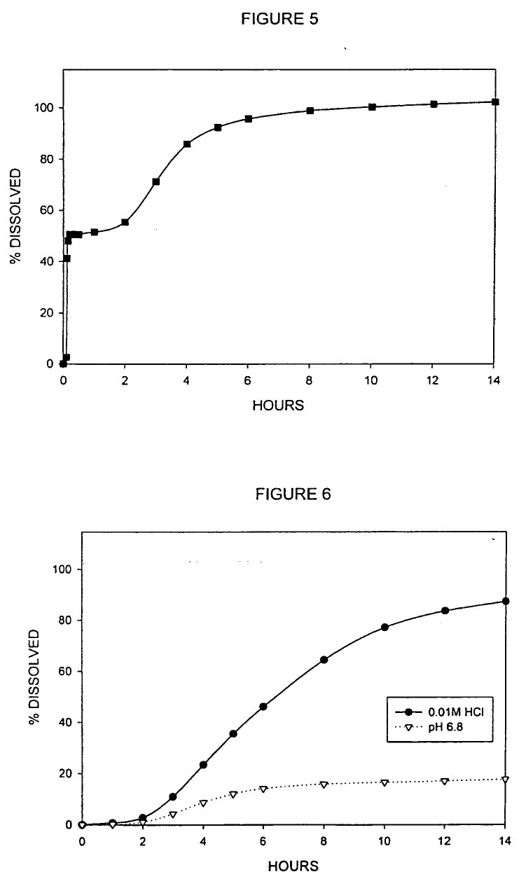

Their dissolution profile in 0.01 M hydrochloric acid obtained as described in example 2, is shown in FIG. 5. The profile parameters in hydrochloric acid are: T₁=2.0 h; T₂=5.0 h.

### COMPARATIVE EXAMPLE 1

### Coated Pellets Containing Zolpidem Hemitartrate

850 g of pellets coated with zolpidem hemitartrate of example 1 were coated in a GPCG1 fluid bed coater-dryer with the following solution

| | | |
|---|---|---|
| ammonio methacrylate | 11.41% | 129.6 g |
| copolymer Type B ¹ | | |
| ammonio methacrylate | 0.92% | 10.5 g |
| copolymer Type A ² | | |
| triethyl citrate ³ | 1.37% | 15.6 g |
| isopropanol | 51.78% | 588.0 g |
| acetone | 34.52% | 392.0 g |

¹ Eudragit ® RS100 commercialized by Rohm Pharma
² Eudragit ® RL100 commercialized by Rohm Pharma
³ Eudraflex ® commercialized by Rohm Pharma

After drying, in a ventilated oven at 35° C. for 24 hours the dissolution profile of the pellets was measured in 0.01 M hydrochloric acid and in pH 6.8, 0.02M phosphate buffer containing 0.1 M sodium chloride as described in example 2. The profiles are shown in FIG. 6. Prolonged dissolution (over about 12 hours) was obtained at 0.01 M hydrochloric acid, but the release rate was very slow at pH 6.8.

### COMPARATIVE EXAMPLE 2

### Coated Pellets Containing Zolpidem Hemitartrate and Tartaric Acid

735 g of nonpareil beads 16/18 mesh were coated using a suspension with the following composition:

| | | |
|---|---|---|
| tartaric acid | 10% | 58.4 g |
| povidone K30 ¹ | 2% | 11.5 g |
| ethanol 95% | 88% | 505.0 g |

¹ Kollidon ® commercialized by BASF

The coated pellets were then coated in a GPG1 fluid bed coater-dryer with the following solution:

| | | |
|---|---|---|
| zolpidem hemitartrate | 11.54% | 78.125 g |
| povidone K30 ² | 11.54% | 78.125 g |
| ethanol | 76.92% | 520.8 g |

² Kollidon ® commercialized by BASF

748 g of the zolpidem hemitartrate-tartaric acid-coated beads were then coated with the following solution.

| | | |
|---|---|---|
| ammonio methacrylate | 11.40% | 62.5 g |
| copolymer Type B ³ | | |
| ammonio methacrylate | 0.93% | 5.1 g |
| copolymer Type A ⁴ | | |
| triethyl citrate ⁵ | 1 37% | 7 5 g |
| isopropanol | 51.80% | 283.5 g |
| acetone | 34.50% | 189.0 g |

³ Eudragit ® RS100 commercialized by Rohm Pharma
⁴ Eudragit ® RL100 commercialized by Rohm Pharma
⁵ Eudraflex ® commercialized by Rohm Pharma

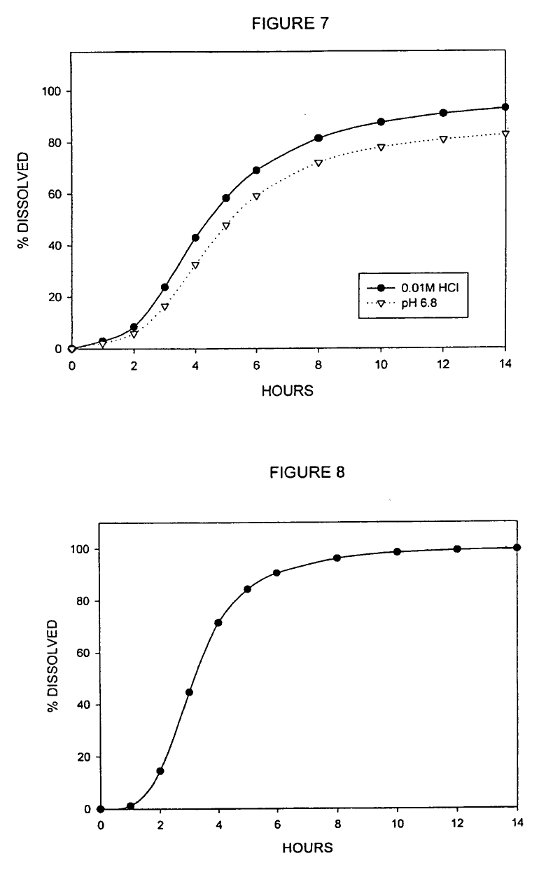

After drying, in a ventilated oven at 35° C. for 24 hours the dissolution profile of the pellets were measured in 0.01 M hydrochloric acid and in a pH 6.8 0.02M phosphate buffer containing 0.1 M sodium chloride as described in example 2. The profiles are shown in FIG. 7. Dissolution was prolonged and independent of pH.

These two comparative examples show that the delayed release pellets comprising acid present a profile of dissolution independent of the pH and that the addition of a cationic surfactant to the tablet core increases the release rate and extent of release at both acid and neutral pH.

### EXAMPLE 5

### Coated Pellets

Delayed release pellets containing zolpidem tartrate, tartaric acid, and cocamidopropylbetaine as an amphoteric surfactant.

1000 g of nonpareil beads 16/18 mesh were coated using a suspension with the following composition, in a GPCG1 fluid bed coater-dryer:

| | | |
|---|---|---|
| tartaric acid | 5.0% | 78.0 g |
| Cocamidopropylbetaine ¹ | 2.5% | 39.0 g |
| Povidone VA 64 ² | 5.0% | 78.0 g |
| Talc | 5.0% | 78.0 g |
| purified water | 41.25% | 643.5 g |
| ethanol 95° | 41.25% | 643.5 g |

¹ Amonyl ® 380LC commercialized by Seppic
² Kollidon ® VA 64 commercialized by BASF

The pellets were then loaded with zolpidem tartrate by coating with the following solution:

| | | |
|---|---|---|
| zolpidem tartrate | 8.3% | 78 g |
| Povidone VA 64 ³ | 8.3% | 78 g |
| ethanol 95° | 83.4% | 784 g |

³ Kollidon ® VA 64 commercialized by BASF

Finally 1000 g of the pellets were coated using a polymer solution of the following composition:

| | | |
|---|---|---|
| ammonio methacrylate | 11.40% | 83.4 g |
| copolymer Type B ⁴ | | |
| ammonio methacrylate | 0.93% | 6.8 g |
| copolymer Type A ⁵ | | |
| triethyl citrate ⁶ | 1.37% | 10 g |
| isopropanol | 51.80% | 379 g |
| acetone | 34.50% | 252 g |

⁴ Eudragit ® RS100 commercialized by Rohm Pharma
⁵ Eudragit ® RL100 commercialized by Rohm Pharma
⁶ Eudraflex ® commercialized by Rohm Pharma

After drying in a ventilated oven, at 30° C. for 16 hours the dissolution profile of the pellets in 0.01 M hydrochloric acid was measured, using the method described in the European Pharmacopoeia, with the rotating paddle apparatus, at a stirring speed of 100 rpm. Dissolution medium was 900 ml, 0.01 M hydrochloric acid at 37° C.±0.5° C. The amount of zolpidem dissolved was measured by UV spectrophotometry at 310 nm. The dissolution curve obtained is shown in FIG. 8.

### EXAMPLE 6

### Tablet Containing Coated Delayed Release Pellets Containing 6 mg Zolpidem Hemitartrate within a Fast-Disintegrating Matrix Containing 6.5 mg Zolpidem Hemitartrate

Prolonged release coated pellets were manufactured as described in example 3. The pellets were then spray-coated using the same method with a layer of 20% by mass of microcrystalline cellulose. A granulate of the following composition was then prepared, by wet granulation:

| | |
|---|---|
| zolpidem hemitartrate | 3.0% |
| lactose | 20.0% |
| microcrystalline cellulose ¹ | 68.0% |
| hydroxypropylmethylcellulose 606 | 3.0% |
| crospovidone ² | 5.0% |
| magnesium stearate | 1.0% |

¹ Avicel ®, commercialized by FMC
² Kollidon ® CL, commercialized by BASF

Pellets and granulate were mixed and compressed into tablets using a rotary press. Each tablet contained 130 mg pellets and 217 mg of the granulate.

What is claimed is:

1. A pharmaceutical composition comprising a short acting hypnotic or a salt thereof adapted to release the short acting hypnotic over a predetermined time period, according to an in vitro profile of dissolution when measured in a rotating paddle apparatus in aqueous buffer at 37° C., comprising two release pulses, the first being immediate, having a maximum duration of 30 minutes, and the second being delayed by a fixed time of between 50 and 200 minutes after the administration, and the delayed second release pulse lasting between 30 and 200 minutes.

2. The pharmaceutical composition according to claim 1, wherein the first pulse has a duration of 20 minutes.

3. The pharmaceutical composition according to claim 1 wherein the fixed time is between 50 and 175 minutes.

4. The pharmaceutical composition according to claim 3 wherein the fixed time is between 60 and 150 minutes.

5. The pharmaceutical composition according to claim 1 wherein 40 to 70% of the total amount of the short acting hypnotic is released during the immediate release pulse.

6. The pharmaceutical composition according to claim 1 wherein the delayed release pulse lasts between 50 and 150 minutes.

7. The pharmaceutical composition according to claim 1 wherein the time for release of 85% of the total amount of the short acting hypnotic is between 2 and 6 hours.

8. The pharmaceutical composition containing a short acting hypnotic or a salt thereof, according to claim 1 comprising two kinds of pharmaceutical entities: one immediate release entity and one delayed release entity.

9. The pharmaceutical composition according to claim 8 as a dosage form selected from the group consisting of capsules, tablets, multilayer tablets, multicoated tablets.

10. The pharmaceutical composition according to claim 8 as a capsule comprising one or more immediate release tablets and one or more delayed release tablets.

11. The pharmaceutical composition according to claim 8 as a capsule comprising a mixture of delayed release particles and immediate release particles.

12. The pharmaceutical composition according to claim 8 as a capsule comprising a mixture of delayed release particles and an immediate release powder.

13. The pharmaceutical composition according to claim 8 as a tablet comprising a number of delayed release coated pellets comprising the short-acting hypnotic imbedded in a matrix.

14. The pharmaceutical composition according to claim 10 wherein the delayed release tablets are coated with at least one ammonio methacrylate copolymer and the tablet core contains a cationic or zwitterionic surfactant.

15. The pharmaceutical composition according to claim 14 wherein the cationic surfactant is selected from the group consisting of trimethyl-dimyristoyl-ammonium propionate, dimethyl-dioctadecyl-ammonium bromide, trimethyl-cetyl-ammonium bromide, dimethyl-didodecyl-ammonium bromide, benzalkonium chloride, cetylpyridinium chloride and cetrimide and the zwitterionic surfactant is selected from the group consisting of N-alkylbetaines, C-alkylbetaines, N-alkylamidobetaines, N-alkylglycines, phosphatidylcholines and lecithins.

16. The pharmaceutical composition according to claim 8 wherein the immediate release entity and the prolonged release entity are administered simultaneously but separately.

17. The pharmaceutical composition according to claim 8 wherein the delayed release entity comprises a pharmaceutically acceptable organic acid selected from the group consisting of tartaric, malic, fumaric, lactic, citric, adipic or succinic acid and their salts, in the form of racemates or isomers.

18. The pharmaceutical composition according to claim 1 wherein the short acting hypnotic belongs to the therapeutic classes of benzodiazepines, cyclopyrrolones, pyrazolopyrimidines, phenothiazines or imidazopyridines.

19. The pharmaceutical composition according to claim 18 wherein the short acting hypnotic is chosen from triazolam, temazepam, brotizolam, zopiclone, (R)-zopiclone, zaleplon, alimemazine, zolpidem and pharmaceutically acceptable salts thereof.

20. The pharmaceutical composition according to claim 19 wherein the short acting hypnotic is zolpidem or a pharmaceutically acceptable salt thereof.

21. The pharmaceutical composition according to claim 2 wherein the fixed time is between 60 and 150 minutes.

22. The pharmaceutical composition according to claim 21 wherein the second pulse lasts between 50 and 150 minutes.

23. The pharmaceutical composition according to claim 22 wherein the 40 to 70% of the total amount of short-acting hypnotic is released during the immediate release pulse.

24. The pharmaceutical composition according to claim 23 wherein the time for release of 85% of the total amount of short-acting hypnotic is between 2 and 6 hours.

25. The pharmaceutical composition according to claim 13 wherein the matrix comprises the short-acting hypnotic.

26. The pharmaceutical composition according to claim 13 wherein immediate release non-coated pellets are mixed with delayed release coated pellets.

27. The pharmaceutical composition according to claim 13 wherein the delayed release coated pellets are further coated with a layer comprising the short-acting hypnotic imbedded in a matrix free from said short-acting hypnotic.

28. The pharmaceutical composition according to claim 13 as a tablet comprising one or more layers containing the delayed release pellets in a matrix free from the short-acting hypnotic and one or more layers containing the short-acting hypnotic in an immediate release matrix.

29. The pharmaceutical composition according to claim 11 wherein the delayed release particles are coated with a mixture containing at least one ammonio methacrylate copolymer and the core contains a cationic or zwitterionic surfactant.

30. The pharmaceutical composition according to claim 13 wherein the delayed release pellets are coated with at least one ammonio methacrylate copolymer and the core contains a cationic or zwitterionic surfactant.

31. The pharmaceutical composition according to claim 25 wherein the delayed release pellets are coated with at least one ammonio methacrylate copolymer and the core contains a cationic or zwitterionic surfactant.

32. The pharmaceutical composition according to claim 26 wherein the delayed release pellets are coated with at least one ammonio methacrylate copolymer and the core contains a cationic or zwitterionic surfactant.

33. The pharmaceutical composition according to claim 27 wherein the delayed release pellets are coated with at least one ammonio methacrylate copolymer and the core contains a cationic or zwitterionic surfactant.

34. The pharmaceutical composition according to claim 28 wherein the delayed release pellets are coated with at least one ammonio methacrylate copolymer and the core contains a cationic or zwitterionic surfactant.

35. The pharmaceutical composition according to claim 29 wherein the cationic surfactant is selected from the group consisting of trimethyl-dimyristoyl-ammonium propionate, dimethyl-dioctadecyl-ammonium bromide, trimethyl-cetyl-ammonium bromide, dimethyl-didodecyl-ammonium bromide, benzalkonium chloride, cetylpyridinium chloride and cetrimide and the zwitterionic surfactant is selected from the group consisting of N-alkylbetaines, C-alkylbetaines, N-alkylamidobetaines, N-alkylglycines, phosphatidylcholines and lecithins.

36. The pharmaceutical composition according to claim 30 wherein the cationic surfactant is selected from the group consisting of trimethyl-dimyristoyl-ammonium propionate, dimethyl-dioctadecyl-ammonium bromide, trimethyl-cetyl-ammonium bromide, dimethyl-didodecyl-ammonium bromide, benzalkonium chloride, cetylpyridinium chloride and cetrimide and the zwitterionic surfactant is selected from the group consisting of N-alkylbetaines, C-alkylbetaines, N-alkylamidobetaines, N-alkylglycines, phosphatidylcholines and lecithins.

37. The pharmaceutical composition according to claim 31 wherein the cationic surfactant is selected from the group consisting of trimethyl-dimyristoyl-ammonium propionate, dimethyl-dioctadecyl-ammonium bromide, trimethyl-cetyl-ammonium bromide, dimethyl-didodecyl-ammonium bromide, benzalkonium chloride, cetylpyridinium chloride and cetrimide and the zwitterionic surfactant is selected from the group consisting of N-alkylbetaines, C-alkylbetaines, N-alkylamidobetaines, N-alkylglycines, phosphatidylcholines and lecithins.

38. The pharmaceutical composition according to claim 32 wherein the cationic surfactant is selected from the group consisting of trimethyl-dimyristoyl-ammonium propionate, dimethyl-dioctadecyl-ammonium bromide, trimethyl-cetyl-ammonium bromide, dimethyl-didodecyl-ammonium bromide, benzalkonium chloride, cetylpyridinium chloride and cetrimide and the zwitterionic surfactant is selected from the group consisting of N-alkylbetaines, C-alkylbetaines, N-alkylamidobetaines, N-alkylglycines, phosphatidylcholines and lecithins.

39. The pharmaceutical composition according to claim 33 wherein the cationic surfactant is selected from the group consisting of trimethyl-dimyristoyl-ammonium propionate, dimethyl-dioctadecyl-ammonium bromide, trimethyl-cetyl-ammonium bromide, dimethyl-didodecyl-ammonium bromide, benzalkonium chloride, cetylpyridinium chloride and cetrimide and the zwitterionic surfactant is selected from the group consisting of N-alkylbetaines, C-alkylbetaines, N-alkylamidobetaines, N-alkylglycines, phosphatidylcholines and lecithins.

40. The pharmaceutical composition according to claim 34 wherein the cationic surfactant is selected from the group consisting of trimethyl-dimyristoyl-ammonium propionate, dimethyl-dioctadecyl-ammonium bromide, trimethyl-cetyl-ammonium bromide, dimethyl-didodecyl-ammonium bromide, benzalkonium chloride, cetylpyridinium chloride and cetrimide and the zwitterionic surfactant is selected from the group consisting of N-alkylbetaines, C-alkylbetaines, N-alkylamidobetaines, N-alkylglycines, phosphatidylcholines and lecithins.

41. The pharmaceutical composition according to claim 15 wherein the core contains cocamidopropylbetaine.

42. The pharmaceutical composition according to claim 35 wherein the core contains cocamidopropylbetaine.

43. The pharmaceutical composition according to claim 36 wherein the core contains cocamidopropylbetaine.

44. The pharmaceutical composition according to claim 37 wherein the core contains cocamidopropylbetaine.

45. The pharmaceutical composition according to claim 38 wherein the core contains cocamidopropylbetaine.

46. The pharmaceutical composition according to claim 39 wherein the core contains cocamidopropylbetaine.

47. The pharmaceutical composition according to claim 40 wherein the core contains cocamidopropylbetaine.

48. The pharmaceutical composition according to claim 29 wherein the delayed release entity comprises a pharmaceutically acceptable organic acid selected from the group consisting of tartaric, malic, fumaric, lactic, citric, adipic or succinic acid and their salts, in the form of racemates or isomers.

49. The pharmaceutical composition according to claim 30 wherein the delayed release entity comprises a pharmaceutically acceptable organic acid selected from the group consisting of tartaric, malic, fumaric, lactic, citric, adipic or succinic acid and their salts, in the form of racemates or isomers.

50. The pharmaceutical composition according to claim 35 wherein the delayed release entity comprises a pharmaceutically acceptable organic acid selected from the group consisting of tartaric, malic, fumaric, lactic, citric, adipic or succinic acid and their salts, in the form of racemates or isomers.

51. The pharmaceutical composition according to claim 36 wherein the delayed release entity comprises a pharmaceutically acceptable organic acid selected from the group consisting of tartaric, malic, fumaric, lactic, citric, adipic or succinic acid and their salts, in the form of racemates or isomers.

52. The pharmaceutical composition according to claim 8 wherein the short acting hypnotic belongs to the therapeutic classes of benzodiazepines, cyclopyrrolones, pyrazolopyrimidines, phenothiazines or imidazopyridines.

53. The pharmaceutical composition according to claim 13 wherein the short acting hypnotic belongs to the therapeutic classes of benzodiazepines, cyclopyrrolones, pyrazolopyrimidines, phenothiazines or imidazopyridines.

54. The pharmaceutical composition according to claim 24 wherein the short acting hypnotic belongs to the therapeutic classes of benzodiazepines, cyclopyrrolones, pyrazolopyrimidines, phenothiazines or imidazopyridines.

55. The pharmaceutical composition according to claim 30 wherein the short acting hypnotic belongs to the therapeutic classes of benzodiazepines, cyclopyrrolones, pyrazolopyrimidines, phenothiazines or imidazopyridines.

56. The pharmaceutical composition according to claim 36 wherein the short acting hypnotic belongs to the therapeutic classes of benzodiazepines, cyclopyrrolones, pyrazolopyrimidines, phenothiazines or imidazopyridines.

57. The pharmaceutical composition according to claim 52 wherein the short acting hypnotic is chosen from triazolam, temazepam, brotizolam, zopiclone, (R)-zopiclone, zaleplon, alimemazine, zolpidem and pharmaceutically acceptable salts thereof.

58. The pharmaceutical composition according to claim 53 wherein the short acting hypnotic is chosen from triazolam, temazepam, brotizolam, zopiclone, (R)-zopiclone, zaleplon, alimemazine, zolpidem and pharmaceutically acceptable salts thereof.

59. The pharmaceutical composition according to claim 54 wherein the short acting hypnotic is chosen from triazolam, temazepam, brotizolam, zopiclone, (R)-zopiclone, zaleplon, alimemazine, zolpidem and pharmaceutically acceptable salts thereof.

60. The pharmaceutical composition according to claim 55 wherein the short acting hypnotic is chosen from triazolam, temazepam, brotizolam, zopiclone, (R)-zopiclone, zaleplon, alimemazine, zolpidem and pharmaceutically acceptable salts thereof.

61. The pharmaceutical composition according to claim 56 wherein the short acting hypnotic is chosen from triazolam, temazepam, brotizolam, zopiclone, (R)-zopiclone, zaleplon, alimemazine, zolpidem and pharmaceutically acceptable salts thereof.

62. The pharmaceutical composition according to claim 57 wherein the short acting hypnotic is zolpidem or a pharmaceutically acceptable salt thereof.

63. The pharmaceutical composition according to claim 58 wherein the short acting hypnotic is zolpidem or a pharmaceutically acceptable salt thereof.

64. The pharmaceutical composition according to claim 59 wherein the short acting hypnotic is zolpidem or a pharmaceutically acceptable salt thereof.

65. The pharmaceutical composition according to claim 60 wherein the short acting hypnotic is zolpidem or a pharmaceutically acceptable salt thereof.

66. The pharmaceutical composition according to claim 61 wherein the short acting hypnotic is zolpidem or a pharmaceutically acceptable salt thereof.

67. A pharmaceutical composition comprising a short acting hypnotic or a salt thereof contained in two pharmaceutical entities, one immediate release entity and one delayed release entity, said composition being adapted to release the short acting hypnotic over a predetermined time period according to an in vitro profile of dissolution when measured in a rotating paddle apparatus in 0.01 M hydrochloric acid buffer at 37° C. at a stirring speed of about 50 to 100 rpm, said time period comprising two release pulses, the first pulse being immediate and having a maximum duration of 30 minutes and the second pulse being delayed by a fixed time of between 50 to 200 minutes after administration, the delayed release entity containing a cationic or zwitterionic surfactant and an organic acid and being coated with an ammonio methacrylate copolymer and the delayed release pulse lasting between 30 and 200 minutes, wherein 40 to 70% of the total amount of the short acting hypnotic is released during the immediate release pulse and the time for release of 85% of the total amount of the short acting hypnotic is between 2 and 6 hours.

68. The pharmaceutical composition according to claim 67 wherein the short acting hypnotic is zolpidem or a pharmaceutically acceptable salt thereof.

69. The pharmaceutical composition according to claim 68 wherein the salt of zolpidem is zolpidem hemitartarate.
